(19) United States
(12) Patent Application Publication
(10) Pub. No.: US 2015/0245764 A1
(43) Pub. Date: Sep. 3, 2015

(54) SUBJECTIVE EYE REFRACTOMETER AND REFRACTOMETRY METHOD

(71) Applicants: SHENZHEN MOPTIM IMAGING TECHNIQUE CO., LTD., Shenzhen,Guangdong (CN); SHENZHEN CENTAINN TECHNOLOGY CO., LTD, Shenzhen,Guangdong (CN)

(72) Inventors: Shuguang GUO, Shenzhen (CN); Hui WANG, Shenzhen (CN); Peng LI, Shenzhen (CN); Xiaoxiang ZHU, Shenzhen (CN); Lei WU, Shenzhen (CN); Dexing ZHANG, Shenzhen (CN)

(21) Appl. No.: 14/366,668

(22) PCT Filed: Nov. 27, 2013
(86) PCT No.: PCT/CN2013/087906

§ 371 Date: Jun. 18, 2014

(30) Foreign Application Priority Data
May 29, 2013 (CN) 201310207478.7

(51) Int. Cl. A61B 3/032; A61B 3/00

(52) U.S. Cl. CPC A61B 3/032; A61B 3/0075

(57) ABSTRACT

A subjective eye refractometer includes an imaging lens and a target, the front surface of the cornea of a testee is located at a focal point of the imaging lens, the target and an eye of the testee are respectively located on two sides of the imaging lens along an optical axis of the imaging lens, and the target can move forward and backward along the optical axis by means of setting, and the subjective eye refractometer is marked with a value D₁ for identifying a spherical refractive-power along a movement path of the target, a movement position x is obtained by subtracting a focal distance f₀ from an object distance of the target, when the testee sees the target clearly, the spherical refractive-power of glasses to be worn on the eyes is the corresponding spherical refractive-power D₁ at the movement position x of the target. The invention further discloses a subjective refractometry method.

This application claims the benefit of priority to CHINA Patent Application No. 201310207478.7 filed with the Chinese Patent Office on May 29, 2013 and entitled “SUBJECTIVE EYE REFRACTOMETER AND REFRACTOMETRY METHOD”, the contents of which are incorporated herein by reference in their entirety.

### FIELD OF THE INVENTION

The present invention relates to a human eye refractometry technology, and in particular, to a subjective eye refractometer and a refractometry method.

### BACKGROUND OF THE INVENTION

Examination of using a visual acuity chart to check eyesight is to check the acuity of an eye. Refractometry examination is to examine the refraction state of an eye. Although the two examinations have certain relevance, but purposes of the two examinations are different, and the two cannot replace each other. Using a traditional visual acuity chart to test human eyesight is simple in device, easy to operate, and has been popularized to common families. However, a traditional refractometry device has a complex structure, needs to be operated by a doctor, and is expensive. Consequently, the traditional refractometry device can hardly be promoted in families and communities, let alone to use the traditional refractometry device as an instrument for self-examination.

### SUMMARY OF THE INVENTION

An objective of the present invention is to overcome the disadvantages of the prior art and to provide a subjective eye refractometer which is simple in structure, convenient in operation, and low in cost.

Another objective is to provide a subjective refractometry method which is simple, convenient, and low in cost.

In order to achieve the foregoing objectives, the present invention employs the following technical solutions:

A subjective eye refractometer includes at least one imaging lens and a target, where the front surface of the cornea of a testee is located at a focal point of the imaging lens, the target and an eye of the testee are respectively located on two sides of the imaging lens along an optical axis of the imaging lens, and the target is capable of moving forward and backward along the optical axis of the imaging lens by means of setting, and the subjective eye refractometer is marked with a value for identifying a spherical refractive-power along a movement path of the target, and a spherical refractive-power D₁ and a movement position x satisfy the following formula:

D₁=x/f₀²,
where the movement position x is obtained by subtracting a focal distance f₀ from an object distance of the target, and
when the eye of the testee sees the target clearly, the spherical refractive-power of the eye of the testee is the corresponding spherical refractive-power D₁ at the movement position x of the target.

A subjective eye refractometer includes an imaging lens and a target, where the front surface of the cornea of a testee is located at a focal point of the imaging lens, an observation plane of the target is provided with signs extending to a plurality of directions, the target and an eye of the testee are respectively located on two sides of the imaging lens along an optical axis of the imaging lens, and the target is capable of moving forward and backward along the optical axis of the imaging lens by means of setting, and the subjective eye refractometer is marked with a value D₁ for identifying a spherical refractive-power along a movement path of the target, a spherical refractive-power D₁ and a movement position x satisfy the following formula:

D₁=x/f₀²;
where the movement position x is obtained by subtracting a focal distance f₀ from an object distance of the target, and
if the eye of the testee sees all the signs clearly, the spherical refractive-power of the eye of the testee is the corresponding spherical refractive-power D₁ at the movement position x of the target, and a cylindrical refractive power of the eye of the testee is 0;
if the eye of the testee fails to see all the signs clearly, the spherical refractive-power and a cylindrical refractive power D_{c} of the eye of the testee are measured in the following manners:
a refractive power of a first movement position of a sign extending to a direction that may be seen by the eye of the testee clearly is: D₁=x/f₀²;
a refractive power of a second movement position of another sign that is perpendicular to the former sign and may be seen by the eye of the testee clearly is: D₃=x₃/f₀²;
if D₁ is the spherical refractive-power, the cylindrical refractive power D_{c}=D₃−D₁, and the axial direction is the direction of the clear sign corresponding to x₃; and
if D₃ is the spherical refractive-power, the cylindrical refractive power D_{c}=D₁−D₃, and the axial direction is the direction of the clear sign corresponding to x.

The shape of the sign the target may be: all endpoints gathered at a same center point, and a plurality of divergence lines diverging to all rounds. More specifically, the form may be as follows:
the target has a plurality of divergence lines diverging uniformly from a center to periphery, and the plurality of divergence lines diverges uniformly within a circle of 360 degrees; or
the target has a plurality of divergence lines diverging uniformly from a center to periphery, and the plurality of divergence lines diverges uniformly within a circle of 360 degrees, and an end of each divergence line diverging externally is marked with an angle, where one or more of the divergence lines serve as a reference and are marked with a reference angle of 0 degree or 180 degrees, and an angle marked on another divergence line is an included angle of the another divergence line relative to the reference divergence line.

A subjective eye refractometer includes an imaging lens, a cylindrical lens, and a target, where the front surface of the cornea of a testee is located at a focal point of the imaging lens, the focal distance of the cylindrical lens is f₂. An observation plane of the target is provided with signs extending to a plurality of directions. The target, the cylindrical lens, the imaging lens, and an eye of the testee are arranged along an optical axis of the imaging lens in sequence. The target and the cylindrical lens can move forward and backward along the optical axis of the imaging lens. The subjective eye refractometer is marked with the value for identifying a spherical refractive-power along a movement path of the target, and the movement path of the cylindrical lens is marked with a value for identifying a cylindrical refractive power.
a spherical refractive-power D₁ and a movement position x satisfy the following formula:

D₁=x/f₀²
the movement position x is obtained by subtracting a focal distance f₀ from an object distance of the target.
if the eye of the testee can see all the signs clearly when the cylindrical lens and the target are overlapped and move together, the spherical refractive-power of the eye of the testee is the corresponding spherical refractive-power D₁ at the movement position x of the target, and the cylindrical refractive power of the eye of the testee is 0;

if the eye of the testee fails to see all the signs clearly when the cylindrical lens and the target are overlapped and move together, the spherical refractive-power and a cylindrical refractive power D_{c} of the eye of the testee are measured in the following manners:
a refractive power of a first movement position of a sign extending to a direction that may be seen by the eye of the testee clearly is: D₁=x/f₀²;
a refractive power of a second movement position of another sign that is perpendicular to the former sign and may be seen by the eye of the testee clearly is: D₃=x₃/f₀²;
if the cylindrical lens is a negative cylindrical lens, the spherical refractive-power of the eye of the testee is a spherical refractive-power at a position where the value is greater between the first movement position and the second movement position, and the direction of the cylindrical lens is adjusted at a position where the value is greater, so that the axis of the cylindrical lens is consistent with a direction of a clear sign, and then the cylindrical lens distance y is moved to ensure that all the signs are clear;
if the cylindrical lens is a positive cylindrical lens, the spherical refractive-power of the eye of the testee is a spherical refractive-power at a position where the value is smaller between the first movement position and the second movement position, and the direction of the cylindrical lens is adjusted at a position where the value is smaller, so that the axis of the cylindrical lens is consistent with a direction of a clear sign, and then the cylindrical lens distance y is moved to ensure that all the signs are clear; and
the cylindrical refractive power D_{c} of the eye of the testee and the movement distance y satisfy the following formula:

$${D}_{c} = ( y + \frac{{yf}_{2}}{y - {f}_{2}} ) / {f}_{0}^{2} .$$

The axial direction of the cylindrical lens is an axial position of the cylindrical refractive power.

A subjective refractometry method includes the following steps:
arranging an imaging lens, a target, and an eye of the testee, where the front surface of the cornea of a testee is located at a focal point of the imaging lens, and the target and an eye of the testee are respectively located on two sides of the imaging lens along an optical axis of the imaging lens; and
moving the target forward and backward along the optical axis of the imaging lens, and if the eye of the testee sees the target clearly when the target is at a movement position x, measuring a spherical refractive-power of the eye of the testee by using the following formula:

D₁=x/f₀²;
where the movement position x is obtained by subtracting a focal distance f₀ from an object distance of the target.

A subjective refractometry method includes the following steps:
arranging an imaging lens, a target, and an eye of the testee, where the front surface of the cornea of a testee is located at a focal point of the imaging lens, an observation plane of the target is provided with signs extending to a plurality of directions, and the target and an eye of the testee are respectively located on two sides of the imaging lens along an optical axis of the imaging lens; and
moving the target forward and backward along the optical axis of the imaging lens, and if the eye of the testee sees the target clearly when the target is at a movement position x, measuring a spherical refractive-power of the eye of the testee by using the following formula:

D₁=x/f₀²;
where the movement position x is obtained by subtracting a focal distance f₀ from an object distance of the target, and
a cylindrical refractive power of the eye of the testee is 0;
if the eye of the testee fails to see all the signs clearly, the spherical refractive-power and a cylindrical refractive power D_{c} of the eye of the testee are measured in the following manners:
a refractive power of a first movement position x of a sign extending to a direction that may be seen by the eye of the testee clearly is: D₁=x/f₀²;
a refractive power of a second movement position x₃ of another sign that is perpendicular to the former sign and may be seen by the eye of the testee clearly is: D₃=x₃/f₀²;
if D₁ is the spherical refractive-power, the cylindrical refractive power D_{c}=D₃−D₁, and the axial direction is the direction of the clear sign corresponding to x₃; and
if D₃ is the spherical refractive-power, the cylindrical refractive power D_{c}=D₁−D₃, and the axial direction is the direction of the clear sign corresponding to x.

A subjective refractometry method includes the following steps:
arranging an imaging lens, a cylindrical lens, and a target, where the front surface of the cornea of a testee is located at a focal point of the imaging lens, a focal distance of the cylindrical lens is f₂ , an observation plane of the target is provided with signs extending to a plurality of directions, and the target, the cylindrical lens, the imaging lens, and an eye of the testee are arranged along an optical axis of the imaging lens in sequence; and
overlapping the cylindrical lens and the target and moving them together along the optical axis of the imaging lens, and if the eye of the testee sees all the signs clearly when the target is at a movement position x, measuring a spherical refractive-power of the eye of the testee by using the following formula:

D₁=x/f₀²;
where the movement position x is obtained by subtracting a focal distance f₀ from an object distance of the target; and
a cylindrical refractive power of the eye of the testee is 0;
if the eye of the testee fails to see all the signs clearly when the cylindrical lens and the target are overlapped and move together, the spherical refractive-power and a cylindrical refractive power D_{c} of the eye of the testee are measured in the following manners:
a refractive power of a first movement position of a sign extending to a direction that may be seen by the eye of the testee clearly is: D₁=x/f₀²;
a refractive power of a second movement position of another sign that is perpendicular to the former sign and may be seen by the eye of the testee clearly is: D₃=x₃/f₀²;
if the cylindrical lens is a negative cylindrical lens, the spherical refractive-power of the eye of the testee is a spherical refractive-power at a position where the value is greater between the first movement position and the second movement position, and the direction of the cylindrical lens is adjusted at a position where the value is greater, so that the axis of the cylindrical lens is consistent with a direction of a clear sign, and then the cylindrical lens distance y is moved to ensure that all the signs are clear;
if the cylindrical lens is a positive cylindrical lens, the spherical refractive-power of the eye of the testee is a spherical refractive-power at a position where the value is smaller between the first movement position and the second movement position, and the direction of the cylindrical lens is adjusted at a position where the value is smaller, so that the axis of the cylindrical lens is consistent with a direction of a clear sign, and then the cylindrical lens distance y is moved to ensure that all the signs are clear; and
the cylindrical refractive power D_{c} of the eye of the testee and the movement distance y satisfy the following formula:

$${D}_{c} = ( y + \frac{{yf}_{2}}{y - {f}_{2}} ) / {f}_{0}^{2} .$$

The axial direction of the cylindrical lens is an axial position of the cylindrical refractive power.

The same points between the present invention and a traditional microscope having refraction adjustment lies in that: 1, both of them have a refraction adjustment (or refraction compensation) function; 2, both of them are designed for seeing a target clearly, for an eyepiece, the target is a detail in an imaging surface of an objective lens, and for the present invention, the target may be a figure on one surface.

The difference between the present invention and the traditional eyepiece of a microscope having refraction adjustment and a corresponding advantage lie in that:

1. With respect to an eyepiece having refraction adjustment, a surface needs to be seen clearly is an imaging lens of an objective lens, and the surface cannot be moved. Therefore, refraction compensation needs to be implemented through moving a lens of the eyepiece. Correspondingly, the position of a human eye needs to be moved too. The eye refractometer in the present invention is different, neither the human eye nor the lens needs to be moved, and only the target needs to be moved.

2. With respect to an eyepiece, the foregoing surface is an imaging lens of an objective lens, the surface is a concept, and there is no object corresponding thereto. The eye refractometer of the present invention is different, the target is an object, such as a designed surface having an exquisitely cut radial line.

3. The eyepiece only has a refraction compensation need, and a refraction compensation degree needs not to be marked. Therefore, there is no requirement for the distance between a human eye and a lens, and usually the human eye is close to the lens as much as possible. In the present invention, optometry needs to be performed on the human eye, and in order to implement liner scale of the spherical refractive-power (a refraction degree), a design concept that the surface of the human eye is located at the focal point of the lens is employed.

4. An eyepiece has no astigmatism compensation design, and the present invention may further implement cylindrical refractive power (astigmatism degree) measurement.

Compared with a traditional optometry device, the subjective eye refractometer of the present invention is simple in structure, small in size, and low in cost. The subjective eye refractometer and the subjective refractometry method of the present invention are also convenient to be handled, easy to be used. Common users can use the subjective eye refractometer to perform self-optometry, and the subjective eye refractometer is particularly applicable to simple optometry in situations such as families and communities.

### BRIEF DESCRIPTION OF DRAWINGS

FIG. 1 is a schematic structural and conceptual diagram of an embodiment illustrating a subjective eye refractometer of the present invention;

FIG. 2 is a schematic structural of a preferable embodiment illustrating a subjective eye refractometer of the present invention;

FIG. 3a is a schematic diagram of a target according to an embodiment of the present invention; and

FIG. 3b is a schematic diagram of a target according to another embodiment of the present invention.

### DESCRIPTION DETAILED DESCRIPTION OF ILLUSTRATED EMBODIMENTS

The present invention is further described in detail with reference to the accompanying drawings and the embodiments.

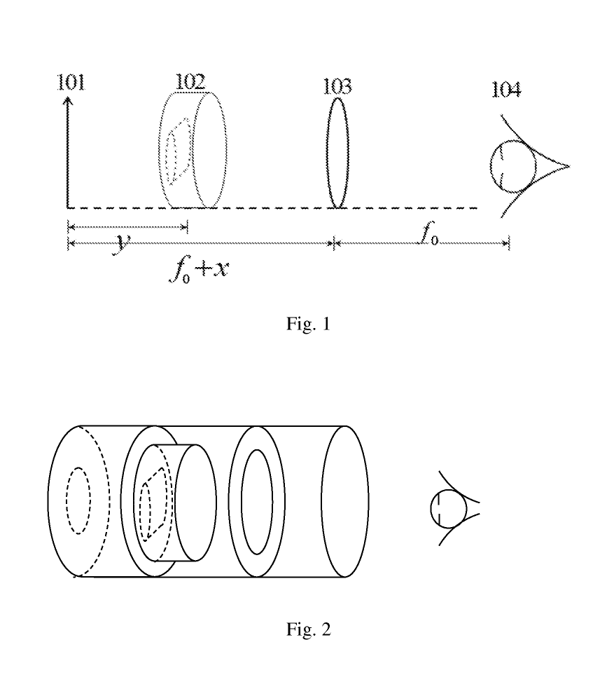

Referring to FIG. 1, in an embodiment, a subjective eye refractometer includes an imaging lens 103 and a target 101. A front surface of the cornea of an eye of a testee 104 is located at a focal point of the imaging lens 103. The target 101 and the eye of the testee 104 are respectively located on two sides of the imaging lens 103 along an optical axis of the imaging lens 103. The target 101 is capable of moving forward and backward along the optical axis of the imaging lens 103 by means of setting. The subjective eye refractometer is marked with a value for identifying the spherical refractive-power D₁ along the movement position x of the target 101 (not illustrated in the figure).

It should be noted that, the imaging lens according to the present invention is not limited to a single lens, which may be a lens combination of a plurality of lens.

As illustrated in FIG. 1, in another embodiment, a subjective eye refractometer includes an imaging lens 103, a cylindrical lens 102, and a target 101. A front surface of the cornea of an eye of a testee 104 is located at a focal point of the imaging lens 103. A focal distance of the cylindrical lens 102 is f₂. An observation plane of the target 101 is provided with signs extending to a plurality of directions (not illustrated in the figure). The target 101, the cylindrical lens 102, the imaging lens 103, and an eye of the testee are arranged along an optical axis of the imaging lens 103 in sequence. The target and the cylindrical lens 102 are capable of moving forward and backward along an optical axis of the imaging lens. The subjective eye refractometer is marked with a value D₁ for identifying the spherical refractive-power D₁ along a movement path of the target 101, and the subjective eye refractometer correspondingly is marked with a value for identifying a cylindrical refractive power along the movement path of the cylindrical lens 102 (not illustrated in the figure).

The value may be a value of a spherical refractive-power or a cylindrical refractive power at a pre-calculated corresponding position, and may also be a intermediate value used to calculate the spherical refractive-power or the cylindrical refractive power.

It should be noted that, the cylindrical lens according to the present invention is not limited to a single cylindrical lens, which may also be a lens combination of a plurality of lens to achieve a cylindrical lens function.

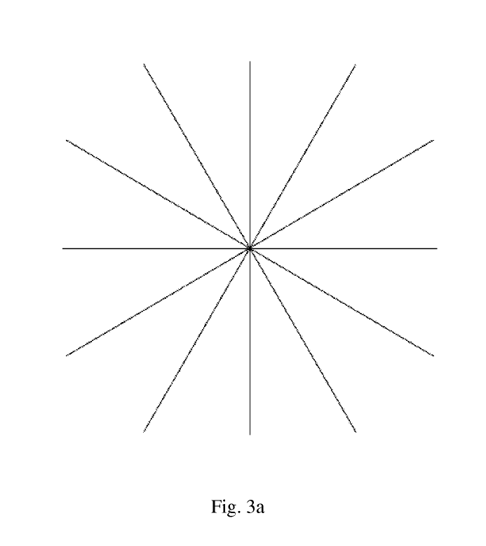

As illustrated in FIG. 2, in another embodiment, a subjective eye refractometer includes an imaging lens 103, a cylindrical lens 102, a target 101, a first sleeve 111, a second sleeve 112, and a third sleeve 113. A front surface of an eye of a testee 104 is located at a focal point of the imaging lens 103. A focal distance of the cylindrical lens 102 is f₂. An observation plane of the target 101 is provided with signs extending to a plurality of directions. The target 101, the cylindrical lens 102, the imaging lens 103, and an eye of the testee 104 are arranged along an optical axis of the imaging lens 103 in sequence. The imaging lens 103 is fixed on the first sleeve 111, and the target 101 is fixed on the second sleeve 112. The second sleeve 112 is capable of sliding on the first sleeve 111. A distance from the sign to the imaging lens 103 is changeable by adjusting the second sleeve 112. The cylindrical lens 102 is fixed on the third sleeve 113. The third sleeve 113 slides on the first sleeve 111 together with the second sleeve 112 when the second sleeve 112 slides. The third sleeve 113 is capable of sliding on the second sleeve 112 and rotating by taking a sleeve axis as a rotation axis.

Lighting may be provided at the rear of a lens cone, and a light source may be disposed at the rear of the lens cone by using natural light (not illustrated in the figure). The first sleeve 111 may be marked with a value for indicating the degree of the spherical refractive-power. The second sleeve 112 may be marked with a value for indicating the degree of the cylindrical refractive power.

In another embodiment, the cylindrical lens 102 of the subjective eye refractometer of the former embodiment and the third sleeve 113 may also be omitted.

Measurement Principles

Spherical refractive-power measurement:

The focal distance of the imaging lens 103 is f₀. The human eye of the testee is located at the position of the focal distance of the imaging lens 103. The object distance of the target 101 relative to the imaging lens 103 is f₀+x, where x is a movement distance of the target, that is, the target is moved to the position, and the human eye can see the target clearly. The image distance is v.

The following may be obtained according to the object-image relationship:

$$\frac{1}{{f}_{0} + x} + \frac{1}{v} = \frac{1}{{f}_{0}}$$

The spherical refractive-power D₁ of a corresponding lens is

$$\frac{1}{v - {f}_{0}} = {D}_{1}$$

bring it to the foregoing formula

$$\frac{1}{{f}_{0} + x} + \frac{1}{{f}_{0} + \frac{1}{{D}_{1}}} = \frac{1}{{f}_{0}}$$

D₁=x/f₀² may be deduced.

That is, through the foregoing formula, the spherical refractive-power of the lens may be obtained from the movement distance of the target, and the movement distance of the target can form a linear relationship with the spherical refractive-power.

Cylindrical refractive power degree measurement (by using cylindrical lens):

For a cylindrical lens 102, the focal distance thereof is f₂.

The object distance of the target 101 relative to the cylindrical lens 102 is y (that is, the movement distance of the cylindrical lens relative to the target 101). The image distance of the image of the target 101 is v₁, and the object-image relationship is:

$$\frac{1}{y} + \frac{1}{{v}_{1}} = \frac{1}{{f}_{2}}$$

An image space of the target 101 relative to the cylindrical lens 102 is an object space of the imaging lens 103, the object space distance thereof is f₁+x−y−v₁, the image space distance thereof is v₂, and an object-image relationship is:

$$\frac{1}{{f}_{1} + x - y - {v}_{1}} + \frac{1}{{v}_{2}} = \frac{1}{{f}_{0}}$$

D₂ is the spherical refractive-power perpendicular to the axis direction of the cylindrical lens;

$$\frac{1}{{v}_{2} - {f}_{2}} = {D}_{2}$$

The following may be obtained with reference to the foregoing formula

$$\begin{matrix}{v}_{1} = 1 / ( \frac{1}{{f}_{2}} - \frac{1}{y} ) \\ = \frac{{yf}_{2}}{y - {f}_{2}}\end{matrix}$$

$${D}_{1} = x / {f}_{0}^{2}$$

$$\begin{matrix}{D}_{2} = \frac{x - y - {v}_{1}}{{f}_{0}^{2}} \\ = \frac{x - y - \frac{{yf}_{2}}{y - {f}_{2}}}{{f}_{0}^{2}}\end{matrix}$$

D_{c} is a cylindrical refractive power degree, that is, cylindrical refractive power

$$\begin{matrix}{D}_{c} = {D}_{1} - {D}_{2} \\ = ( y + \frac{{yf}_{2}}{y - {f}_{2}} ) / {f}_{0}^{2}\end{matrix}$$

The cylindrical refractive power may also be measured by omitting the embodiment of the cylindrical lens.

### Embodiment 1

A target 101 may be a picture, which has streak lines of different directions or divergence lines similar to a traditional astigmatic chart, and the like. Divergence lines (the target is marked with a symbol of “ [symbol] ”, divergence line shape) are taken as an example in the following description.

When the target is moved to a position x and lines of all directions of the target “ [symbol] ”0 are clear at the same time, the spherical refractive-power of the testee, that is, the spherical refractive-power is D₁=x/f₀². At this time, the testee has no astigmatism or the astigmatism is little and may be omitted. The cylindrical refractive power, that is, the cylindrical refractive power is 0.

When lines of all directions of the target “ [symbol] ” are not clear at the same time, there are two positions where two divergence lines that are perpendicular to each other are clear.

Referring to FIG. 1, the target 101 moves forward and backward during measurement. If two positions where a line of a direction of the divergence lines may be seen clearly occurs, and the divergence lines of two directions are perpendicular to each other;
with respect to a first position, a calculated spherical refractive-power is: D₁=x/f₀²;
with respect to a second position, a calculated spherical refractive-power is :D₃=x₃/f₀²;
if D₁ is the spherical refractive-power, D₃−D₁ D₁=D₁ is the cylindrical refractive power, and the axial direction is the direction of the divergence line corresponding to x₃; and
if D₃ is the spherical refractive-power, D₁−D₃ is the cylindrical refractive power, and the axial direction is the direction of the divergence line corresponding to x.

In this embodiment, spherical refractive-power and a cylindrical refractive power measurement may be implemented without the cylindrical lens.

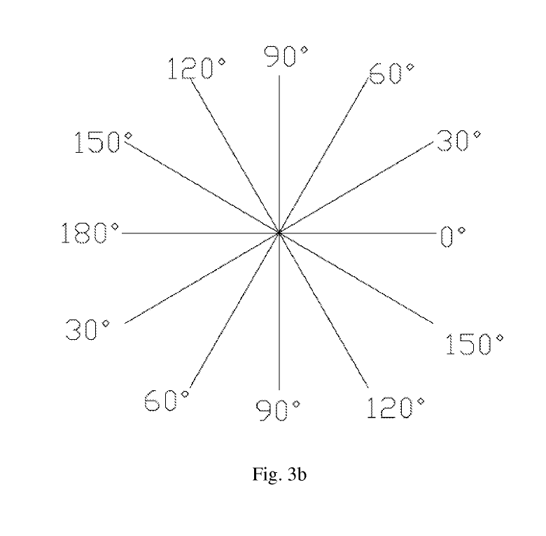

Referring to FIG. 3a and FIG. 3b, the shape of the sign at the target may be: all endpoints gathered at a same center point, and a plurality of divergence lines diverging to all rounds.

More specifically, as illustrated in FIG. 3a, the shape of the target may be, a plurality of divergence lines diverging uniformly from a center to periphery, and the plurality of divergence lines are disposed uniformly within a circle of 360 degrees.

As illustrated in FIG. 3b, on basis of the target illustrated in FIG. 3a, an end of each divergence line that diverges externally is marked with an angle, where one or more divergence lines serve as a reference, and have a reference angle. For example, two divergence lines in a horizontal direction in FIG. 3b are marked with 0 degree and 180 degrees respectively, and an angle marked on another divergence line is an included angle thereof relative to the two reference divergence lines.

The angle of each two adjacent divergence lines and the angle marked at the divergence line are relevant to the number of divergence lines. For example, in the target illustrated in FIG. 3b, the number of divergence lines is 12, the included angle of each two adjacent divergence lines is 30 degrees, and the angle marked at the divergence line is also changed by taking 30 degrees as a gradient.

The advantage of marking an angle is: when the human eye astigmatism degree is checked, a drum of a subjective eye refractometer is rotated, and when an eye can see a divergence line clearly, a corresponding angle may be found, thereby determining an astigmatism degree conveniently.

The number of divergence lines of the target is unlimited, and there is a plurality of angles corresponding thereto. For example, except angles 0, 30, 60, 90, 120, 150, and 180 of the divergence line illustrated in FIG. 3b, the following may further be included:
0, 45, 90, 135, 180; or
0, 15, 30, 45, 60, 75, 90, 105, 120, 135, 150, 165, 180, and the like.

That is, the number of divergence lines and the angle of adjacent divergence lines may be determined flexibly.

### Embodiment 2

In the same way, a target 101 may be a picture, which has streak lines of different directions or divergence lines similar to a traditional astigmatic chart, and the like. Divergence lines (the target is marked with a symbol of “ [symbol] ”, divergence line shape) are taken as an example in the following description. In this embodiment, a cylindrical lens 102 is used, the cylindrical lens 102 and the target 101 are overlapped and moved forward and backward, and there are two conditions:

When a testee can see all lines in the target “ [symbol] ” clearly at a position x, the spherical refractive-power degree of the testee may be calculated according to the spherical refractive-power computing method, and it may be obtained that the human eye has no astigmatism.

When two positions are moved to and the testee can see one line of two lines that are perpendicular to each other in the target “ [symbol] ” symbol clearly, the spherical refractive-power degree at the two positions may be calculated according to the spherical refractive-power computing method in the foregoing text.

And it may be learned that the human eye has astigmatism, and a cylindrical refractive power measurement may be performed following: If a negative cylindrical lens is adopted, a position where x is a larger value is adjusted to so that one line of the divergence lines is most clear, and the spherical refractive-power degree at that position is the spherical refractive-power of the testee. And then, the direction of the cylindrical lens is adjusted so that the direction of the axis of the cylindrical lens is the same as the direction of the most clear line of the divergence lines, and the cylindrical lens is moved to ensure that all the divergence lines are clear. The movement distance of the cylindrical lens is y, and the cylindrical refractive power may be deduced according to the foregoing formula.

If a positive cylindrical lens is adopted, a position where x is a smaller value is adjusted to so that one line of the divergence lines is most clear, and the spherical refractive-power degree at that position is the spherical refractive-power of the testee. And then, the direction of the cylindrical lens is adjusted so that the direction of the axis of the cylindrical lens is the same as the direction of the most clear line of the divergence lines, and the cylindrical lens is moved to ensure that all the divergence lines are clear. The movement distance of the cylindrical lens is y, and the cylindrical refractive power may be deduced according to the foregoing formula. The axial direction of the cylindrical lens is an axial position of the cylindrical refractive power.

Compared with Embodiment 1, in Embodiment 2, the design of using a cylindrical lens to measure a cylindrical refractive power is adopted, and the obvious advantage lies in that direct marking of a cylindrical refractive power may be implemented directly so as to facilitate reading the measurement result.

Compared with Embodiment 1, the advantage of Embodiment 2 lies in that the device is simpler and the cost is lower.

In the same way, in Embodiment 2, the target may also take the form of the target illustrated in FIG. 3a and FIG. 3b.

### Embodiment 3

Referring to FIG. 2, a specific embodiment is illustrated.

A lens 103 is fixed in a first sleeve 111, and a front surface of a cornea an eye of a testee 104 is located at a focal point of the lens 103. A target 101 may be a picture, which has streak lines of different directions or divergence lines similar to a traditional astigmatic chart, and the like. Divergence lines are taken as an example in the following description. The target 101 is fixed on a second sleeve 112, the second sleeve 112 may slide within the first sleeve 111, and a distance from the target to the lens may be changed by adjusting the second sleeve 112. The cylindrical lens is fixed on the third sleeve 113, and the third sleeve 113 may slide within the second sleeve 112 and rotate by taking a sleeve axis as a rotation axis. A distance of the cylindrical lens and the distance from the cylindrical lens to the lens may be changed by adjusting the third sleeve 113. When the second sleeve 112 is adjusted, the third sleeve 113 slides on the first sleeve 111 together with the second sleeve 112.

The cornea of the testee needs to be located at the focal point position of the imaging lens 103. In this case, a degree (spherical refractive-power) marked at the sleeve is in a linear relationship with the movement distance of the target 101.

When the testee is myopia or hyperopia, the movement of the target 101 may be implemented by rotating the sleeve of a body to ensure that the testee can see the target 101 clearly and the sleeve rotation is stopped. At this time, the spherical refractive-power marked at the sleeve is the myopia degree of the testee.

During measurement, the third sleeve 113 is adjusted first so that the cylindrical lens sticks with the target, that is, the distance from the cylindrical lens to the lens is zero.

At this time, the cylindrical lens makes no contribution to imaging of the target. The initial distance from the target to the lens is the focal distance f₀ of the lens.

At this time, the image of the target is at infinity, and if testee is an emmetropia, the testee can see a clear divergence line.

That is, when the spherical refractive-power of the testee is 0 (normal eyesight) and the target is located at a focal point position of the imaging lens, the testee can see the divergence line of the target 101 clearly.

During measurement, the distance from the target to the lens may be changed by adjusting the second sleeve 112 to ensure that the divergence lines are clear or one direction of the divergence lines is clear. If all directions of the divergence line of the second sleeve 112 are clear at a position, it indicates that the testee has no astigmatism or the astigmatism little and may be omitted. If all directions of the divergence line of the second sleeve 112 are clear at a position, it indicates that the testee has no astigmatism or the astigmatism little and may be omitted. If it is found that only one direction of the divergence line of the second sleeve 112 is clear at a position, another position can surely be found by adjusting the second sleeve 112. At that position, the testee can find that only a divergence line that is perpendicular to the divergence line is clear.

By recording a corresponding movement position of the sleeve, the spherical refractive-power and a cylindrical refractive power may be obtained by checking a pre-calculated value.

### Embodiment 4

Different from the specific embodiment illustrated in FIG. 2, the cylindrical lens 102 of the subjective eye refractometer and the third sleeve 113 are omitted. The spherical refractive-power and a cylindrical refractive power may be measured according to the method in Embodiment 1.

Detailed above further describes the present invention with reference to specific and exemplary embodiments, but the specific embodiments of the present invention should not be construed as limited to the description. Any simple deduction or replacement figured out by a person skilled in the art without departing from the inventive concept of the present invention shall fall within the protection scope of the present invention.

What is claimed is:

1. A subjective eye refractometer, comprising at least one imaging lens and a target, wherein the front surface of the cornea of a testee is located at a focal point of the imaging lens, the target and an eye of the testee are respectively located on two sides of the imaging lens along an optical axis of the imaging lens, and the target is capable of moving forward and backward along the optical axis of the imaging lens by means of setting, and the subjective eye refractometer is marked with a value for identifying a spherical refractive-power along a movement path of the target, wherein a spherical refractive-power D₁ and a movement position x satisfy the following formula:
D₁=x/f₀²;
where the movement position x is obtained by subtracting a focal distance f₀ from an object distance of the target; and
when the eye of the testee sees the target clearly, the spherical refractive-power of the eye of the testee is the corresponding spherical refractive-power D₁ at the movement position x of the target.

2. A subjective eye refractometer, comprising an imaging lens and a target, wherein the front surface of the cornea of a testee is located at a focal point of the imaging lens, an observation plane of the target is provided with signs extending to a plurality of directions, the target and an eye of the testee are respectively located on two sides of the imaging lens along an optical axis of the imaging lens, and the target is capable of moving forward and backward along the optical axis of the imaging lens by means of setting, and the subjective eye refractometer is marked with a value for identifying a spherical refractive-power along a movement path of the target, a spherical refractive-power D₁ and a movement position x satisfy the following formula:
D₁=x/f₀²;
where the movement position x is obtained by subtracting a focal distance f₀ from an object distance of the target, and
when the eye of the testee sees all the signs clearly, the spherical refractive-power of the eye of the testee is the corresponding spherical refractive-power D₁ at the movement position x of the target, and a cylindrical refractive power of the eye of the testee is 0;
if the eye of the testee fails to see all the signs clearly, the spherical refractive-power and a cylindrical refractive power D_{c} of the eye of the testee are measured in the following manners:
a refractive power of a first movement position of a sign extending to a direction that may be seen by the eye of the testee clearly is: D₁=x/f₀²;
a refractive power of a second movement position of another sign that is perpendicular to the former sign and may be seen by the eye of the testee clearly is: D₃=x₃/f₀²;
if D₁ is the spherical refractive-power, the cylindrical refractive power D^{c}=D₃−L₁, and the axial direction is the direction of the clear sign corresponding to x₃; and
if D₃ is the spherical refractive-power, the cylindrical refractive power D_{c}=D₁−D₃, and the axial direction is the direction of the clear sign corresponding to x.

3. The subjective eye refractometer according to claim 2, further comprising a first sleeve and a second sleeve, wherein the imaging lens is fixed on the first sleeve, the target is fixed on the second sleeve, the second sleeve is capable of sliding on the first sleeve by means of setting, a distance from the sign to the imaging lens is changeable by adjusting the second sleeve, the value for identifying the spherical refractive-power is marked on the first sleeve or the second sleeve along a sleeve axis.

4. The subjective eye refractometer according to claim 2, wherein the target is in the following forms:
the target has a plurality of divergence lines diverging uniformly from a center to periphery, and the plurality of divergence lines diverges uniformly within a circle of 360 degrees; or
the target has a plurality of divergence lines diverging uniformly from a center to periphery, the plurality of divergence lines diverges uniformly within a circle of 360 degrees, and an end of each divergence line diverging externally is marked with an angle, where one or more of the divergence lines serve as a reference, and marked with a reference angle of 0 degree or 180 degrees, and an angle marked on another divergence line is an included angle of the another divergence line relative to the reference divergence line.

5. A subjective eye refractometer, comprising an imaging lens, a cylindrical lens, and a target, wherein the front surface of the cornea of a testee is located at a focal point of the imaging lens, the focal distance of the cylindrical lens is f₂, an observation plane of the target is provided with signs extending to a plurality of directions, the target, the cylindrical lens, the imaging lens, and an eye of the testee are arranged along an optical axis of the imaging lens in sequence, the target and the cylindrical lens can move forward and backward along the optical axis of the imaging lens, the subjective eye refractometer is marked with the value for identifying a spherical refractive-power along a movement path of the target, and the movement path of the cylindrical lens is marked with a value for identifying a cylindrical refractive power;
a spherical refractive-power D₁ and a movement position x satisfy the following formula:
D₁x/f₀²
the movement position x is obtained by subtracting a focal distance f₀ from an object distance of the target, and
if the eye of the testee can see all the signs clearly when the cylindrical lens and the target are overlapped and move together, the spherical refractive-power of the eye of the testee is the corresponding spherical refractive-power D₁ at the movement position x of the target, and the cylindrical refractive power of the eye of the testee is 0;
if the eye of the testee fails to see all the signs clearly when the cylindrical lens and the target are overlapped and move together, the spherical refractive-power and a cylindrical refractive power D_{c} of the eye of the testee are measured in the following manners:
a refractive power of a first movement position of a sign extending to a direction that may be seen is: D₁=x/f₀²,
a refractive power of a second movement position of another sign that is perpendicular to the former sign and may be seen clearly is: D₃=x₃/f₀²;
if the cylindrical lens is a negative cylindrical lens, the spherical refractive-power of the eye of the testee is a spherical refractive-power at a position where the value is greater between the first movement position and the second movement position, and the direction of the cylindrical lens is adjusted at a position where the value is greater, so that the axis of the cylindrical lens is consistent with a direction of a clear sign, and then the cylindrical lens distance y is moved to ensure that all the signs are clear;
if the cylindrical lens is a positive cylindrical lens, the spherical refractive-power of the eye of the testee is a spherical refractive-power at a position where the value is smaller between the first movement position and the second movement position, and the direction of the cylindrical lens is adjusted at a position where the value is smaller, so that the axis of the cylindrical lens is consistent with a direction of a clear sign, and then the cylindrical lens distance y is moved to ensure that all the signs are clear;
the cylindrical refractive power D_{c} of the eye of the testee and the movement distance y satisfy the following formula:
$${D}_{c} = ( y + \frac{{yf}_{2}}{y - {f}_{2}} ) / {f}_{0}^{2}$$
the axial direction of the cylindrical lens is an axial position of the cylindrical refractive power.

6. The subjective eye refractometer according to claim 5, further comprising: a first sleeve, a second sleeve, and a third sleeve, wherein the imaging lens is fixed on the first sleeve, the target is fixed on the second sleeve, the second sleeve can slide on the first sleeve after setting, the distance from the target to the imaging lens may be changed by adjusting the second sleeve, the cylindrical lens is fixed on the third sleeve, when the second sleeve slides, the third sleeve slides with the second sleeve on the first sleeve, the third sleeve can slide on the second sleeve and rotate by taking the second sleeve axis as a rotation axis, the value for identifying the spherical refractive-power is marked on the first sleeve along the sleeve axial direction, and a value for identifying a cylindrical refractive power is marked on the second sleeve along the sleeve axis.

7. A subjective refractometry method, comprising:
arranging an imaging lens, a target, and an eye of a testee, wherein the front surface of the cornea of a testee is located at a focal point of the imaging lens, and the target and an eye of the testee are respectively located on two sides of the imaging lens along an optical axis of the imaging lens,
moving the target forward and backward along the optical axis of the imaging lens, and if the eye of the testee sees the target clearly when the target is at a movement position x, measuring a spherical refractive-power of the eye of the testee by using the following formula:
D₁=x/f₀²
the movement position x is obtained by subtracting a focal distance f₀ from an object distance of the target.

8. The subjective refractometry method according to claim 7, wherein the target is a figure on a flat surface.

9. A subjective refractometry method, comprising:
arranging an imaging lens, a target, and an eye of the testee, wherein the front surface of the cornea of a testee is located at a focal point of the imaging lens, an observation plane of the target is provided with signs extending to a plurality of directions, and the target and an eye of the testee are respectively located on two sides of the imaging lens along an optical axis of the imaging lens,
moving the target forward and backward along the optical axis of the imaging lens, and if the eye of the testee sees the target clearly when the target is at a movement position x, measuring a spherical refractive-power of the eye of the testee by using the following formula:
D₁=x/f₀²;
where a movement position x is obtained by subtracting a focal distance f₀ from an object distance of the target, and
a cylindrical refractive power of the eye of the testee is 0;
if the eye of the testee fails to see all the signs clearly, the spherical refractive-power and a cylindrical refractive power D_{c} of the eye of the testee are measured in the following manners:
a refractive power of a first movement position x of a sign extending to a direction that may be seen by the eye of the testee clearly is: D₁=x/f₀²;
a refractive power of a second movement position x₃ of another sign that is perpendicular to the former sign and may be seen by the eye of the testee clearly is: D₃=x₃/f₀²;
if D₁ is the spherical refractive-power, the cylindrical refractive power D_{c}=D₃−D₁, and the axial direction is the direction of the clear sign corresponding to x₃; and
if D₃ is the spherical refractive-power, the cylindrical refractive power D_{c}=D₁−D₃, and the axial direction is the direction of the clear sign corresponding to x.

10. A subjective refractometry method, comprising:
arranging an imaging lens, a cylindrical lens, and a target, wherein the front surface of the cornea of a testee is located at a focal point of the imaging lens, a focal distance of the cylindrical lens is f₂, an observation plane of the target is provided with signs extending to a plurality of directions, the target, the cylindrical lens, the imaging lens, and an eye of the testee are arranged along an optical axis of the imaging lens in sequence; and
overlapping the cylindrical lens and the target and moving them together along the optical axis of the imaging lens, and if the eye of the testee sees all the signs clearly when the target is at a movement position x, measuring a spherical refractive-power of the eye of the testee by using the following formula:
D₁=x/f₀²
the movement position x is obtained by subtracting a focal distance f₀ from an object distance of the target, and
the cylindrical refractive power of the eye of the testee is 0;
if the eye of the testee fails to see all the signs clearly when the cylindrical lens and the target are overlapped and move together, the spherical refractive-power and a cylindrical refractive power D_{c} of the eye of the testee are measured in the following manners:
a refractive power of a first movement position of a sign extending to a direction that may be seen is: D₁=x/f₀²;
a refractive power of a second movement position of another sign that is perpendicular to the former sign and may be seen clearly is: D₃=x₃/f₀²;
if the cylindrical lens is a negative cylindrical lens, the spherical refractive-power of the eye of the testee is a spherical refractive-power at a position where the value is greater between the first movement position and the second movement position, and the direction of the cylindrical lens is adjusted at a position where the value is greater, so that the axis of the cylindrical lens is consistent with a direction of a clear sign, and then the cylindrical lens distance y is moved to ensure that all the signs are clear;
if the cylindrical lens is a positive cylindrical lens, the spherical refractive-power of the eye of the testee is a spherical refractive-power at a position where the value is smaller between the first movement position and the second movement position, and the direction of the cylindrical lens is adjusted at a position where the value is smaller, so that the axis of the cylindrical lens is consistent with a direction of a clear sign, and then the cylindrical lens distance y is moved to ensure that all the signs are clear; and
the cylindrical refractive power D_{c} of the eye of the testee and the movement distance y satisfy the following formula:
$${D}_{c} = ( y + \frac{{yf}_{2}}{y - {f}_{2}} ) / {f}_{0}^{2} ;$$
where the axial direction of the cylindrical lens is an axial position of the cylindrical refractive power.
